(19) United States
(12) Patent Application Publication
(10) Pub. No.: US 2017/0343532 A1
(43) Pub. Date: Nov. 30, 2017

(54) MEDICAL DEVICE WITH A TWISTING BUTTON GIVING THE IMPRESSION TO BE PART OF A DISPLAY WINDOW

(71) Applicant: Roche Diabetes Care, Inc., Indianapolis, IN (US)

(72) Inventors: Michel Alain Jean Marie Cadio, Carmel, IN (US); Christoph Gerhard Eisenhardt, Mannheim (DE)

(73) Assignee: Roche Diabetes Care, Inc., Indianapolis, IN (US)

(21) Appl. No.: 15/164,693

(22) Filed: May 25, 2016

(51) Int. Cl. G01N 33/487; H01H 13/50; A61B 5/00; H05K 5/00; A61B 5/145; A61B 5/15; G01N 33/49

(52) U.S. Cl. CPC G01N 33/48785; A61B 5/150274; H01H 13/50; G01N 33/49; H05K 5/0017; A61B 5/14532; A61B 5/742; A61B 5/150358; A61B 2562/166; A61B 2560/0406

(57) ABSTRACT

A medical device includes a housing that has a front face and a side face. The housing defines a window cavity on the front face and a button cavity in the side face. A display is disposed in the housing. The display is aligned in the housing for viewing through the window cavity. A window covers the display in the window cavity, and the display is at least partially visible through the window. The window extends along the front face to the button cavity in the side face. A button element is received in the button cavity. The button element and the window are separate components. The button element has opposing end edges that align with the end edges of the window where the button element and window meet to provide an impression that the window and the button element are integral with one another.

### BACKGROUND

With the advent of electronic home medical devices, there is a desire to have the medical devices be both functional under a wide variety of conditions as well as aesthetically pleasing to consumers. One common usage is home monitoring of blood glucose levels via glucose meters. As a result of their condition, some diabetics experience visual impairment as well as hand dexterity or control issues. If the user improperly reads the glucose meter and/or accidentally hits the wrong button, it may lead to undesirable consequences, such as improper glucose control by the diabetic. Moreover, buttons or other input devices in glucose meters are typically subjected to repeated actuation which can lead to failure of the meter at inopportune times. Thus, there is a need for improvement in this field.

### SUMMARY

Common glucose meters typically are designed from a functional perspective and are commonly aesthetically unappealing. In a customary glucose meter design, the display and the accompanying lens or window protecting the display are framed by the housing on the front of the meter. To provide a more appealing look, a one piece wrap around window design was developed in which the window for the display stretched from the front face of the meter to the side of the meter. The side portion of the window further included an opening for a side button that is used to interact with the meter. Though this wraparound window design provided an aesthetically pleasing glucose meter, it was unexpectedly discovered that this design had some significant drawbacks which made it difficult to produce in a cost effective manner. For example, it created issues with any visual items, such as instructions or decorative items, stretching from the front face of the window to the side. Due to the molding requirements for producing the window, a mold parting line is created generally around the bend where the window transitions from the front face to the side. Any visual items stretching across this parting line would is generally require painting with masking. This masking process typically causes poor yields when a very good appearance quality is required for painted items. Painting with masking also requires the addition of a groove between the painted and unpainted areas. This groove damages the appearance of the smooth flat sections of the window. It was further found that the side opening in the window for the button creates other issues. Given the button extends through the side opening, insertion of the window frame becomes difficult during assembly. Moreover, the overall design of the meter becomes more constrained which in turn results in less robust product assembly. When the bend between the front and side of the window is at an acute angle, mold undercut issues are created. As a result, a more complicated mold is required, such as those requiring collapsible cores or mold inserts. The interior molding would also have to properly align with the exterior molding so as to form the button opening. The mold requirements together would make it difficult to maintain mold tooling because the parting line is now located between two moving parts of the molding. This in turn can result in mold flashing occurring along the parting line of the window during production.

To address these as well as other issues, an integrated interface system was developed to give the impression that the button is part of a front decorative window of a medical device, such as a glucose meter. The meter includes a unique molded button that uses a flexing and twisting action during actuation of the button and has the appearance of being part of the front lens or window of the meter. However, the meter actually utilizes a two-piece construction. Specifically, the button element is in the form of a narrow strip that includes a stiffening rib or beam positioned to face the front of the meter such that it is offset to induce a twisting motion when the button is pressed. The length of the button element generally coincides with the width of the lens so as to give the appearance of a smooth transition between the front facing lens and the side facing button element. The button element further includes a hard point, such as in the form of an alignment rib for locating purposes and retention flanges positioned to keep the button element within the back shell of the housing. One of the flanges includes a button actuator that is used to activate the switch in the meter. When the meter is assembled, the beam of the button element is not visible to the user but is able to facilitate the twisting action of the button element. The stiffness of the rib is selected to create the proper twisting action for applying the proper force to the switch. This design allows the button element to be easily captured between the front and back shells of the housing during assembly. When the front shell snaps onto the back shell, the is button element is trapped in its final position. The resulting design gives the impression that the window has a waterfall type shape without the drawbacks associated with the previously mentioned wrap around window design. For example, complicated mold tooling is not required even when the side of the meter is angled at an acute angle relative to the front face because the window and button element are molded separately. With this unique design, a reliable button actuator is created in an intricate location of the meter. Moreover, it is cost effective because it helps to simplify the mold construction and is simple to assemble. The risk of unintended actuation of the button when holding the meter is also reduced because the wrap around window appearance gives a visual indicator of where to press.

Aspect 1 concerns a medical device. The medical devices includes a housing having a front face and a side face. The housing defines a window cavity in the front face and a button cavity in the side face. A display is disposed in the housing, and the display is aligned in the housing for viewing through the window cavity. A window covers the display in the window cavity. The display is at least partially visible through the window. The window extends along the front face to the button cavity in the side face. The window has opposing end edges. A button element is received in the button cavity. The button element and the window are separate components. The button element has opposing end edges that align with the end edges of the window where the button element and the window meet to provide an impression that the window and the button element are integral with one another.

Aspect 2 concerns the device of any preceding aspect, wherein the button element has an exterior surface. The window has a rounded edge that at least a portion of which is flush with the exterior surface of the button element where the window and button element meet to provide the impression that the window bends from the front face to the side face.

Aspect 3 concerns the device of any preceding aspect, wherein the button element has a button portion that protrudes from the exterior surface of the button element.

Aspect 4 concerns the device of any preceding aspect, wherein the window and the button element have similar visual appearances.

Aspect 5 concerns the device of any preceding aspect, wherein the window and the button element at least where the window and the button element meet have the same color.

Aspect 6 concerns the device of any preceding aspect, wherein the button element includes a stiffening structure asymmetrically positioned along a longitudinal axis of the button element to promote a twisting motion of the button element when pressed.

Aspect 7 concerns the device of any preceding aspect, wherein the stiffening structure includes a stiffening rib positioned proximal to a side of the button element facing the window.

Aspect 8 concerns the device of any preceding aspect, wherein the button element includes an alignment rib extending transverse to the stiffening rib to form a T-shape.

Aspect 9 concerns the device of any preceding aspect, wherein the stiffening structure includes a lattice of ribs defining rib cavities.

Aspect 10 concerns the device of any preceding aspect, wherein the housing includes a button alignment structure defining an alignment groove. The alignment structure of the button element includes an alignment rib received in the alignment groove.

Aspect 11 concerns the device of any preceding aspect, wherein the housing includes a front shell defining the window cavity, and a back shell defining the button cavity. The front shell and the back shell are coupled together to trap the button element in the button cavity.

Aspect 12 concerns the device of any preceding aspect, wherein the button element includes one or more retention flanges engaging the button shell around the button cavity.

Aspect 13 concerns the device of any preceding aspect, further including a switch. The button element includes an actuator foot extending from one of the retention flanges to actuate the switch.

Aspect 14 concerns the device of any preceding aspect, wherein the button element has an interior side that faces an interior of the housing and an exterior side that is opposite to the interior side. The actuator foot extends from the interior side of the button element. The button element has a button portion protruding on the exterior side. The button portion is aligned to be opposite the actuator foot.

Aspect 15 concerns the device of any preceding aspect, wherein a seam between the front shell is aligned with a seam between the window and the button element.

Aspect 16 concerns the device of any preceding aspect, wherein the button element has one or more spacer tabs extending into a seam between the window and the button element.

Aspect 17 concerns the device of any preceding aspect, wherein the housing includes a test strip connector port.

Aspect 18 concerns a medical device. A housing has a side face that defines a button cavity. A button element is retained in the button cavity. The button element has a stiffening rib located at an offset position along the length of the button element to induce a twisting motion in the button when pressed.

Aspect 19 concerns the device of any preceding aspect, wherein the button element includes an alignment rib extending transverse to the stiffening rib to form a T-shape.

Aspect 20 concerns the device of any preceding aspect, wherein the housing includes a button alignment structure defining an alignment groove. The alignment rib is received in the alignment groove.

Aspect 21 concerns the device of any preceding aspect, wherein the button element includes a lattice of ribs defining rib cavities.

Aspect 22 concerns the device of any preceding aspect, wherein the button element includes a first edge and a second edge located opposite to the first edge. The button element has an actuator foot positioned along the first edge at one end of the button element. The actuator foot extends from an interior surface of the button element. The button element has a button portion protruding from an exterior surface at a position aligned with the actuator foot. The stiffening rib extends along the second edge of the button element.

Aspect 23 concerns the device of any preceding aspect, wherein the button element includes one or more retention flanges engaging the housing around the button cavity.

Aspect 24 concerns the device of any preceding aspect, wherein the housing has a front face with a window. The window extends along the front face to the button cavity in the side face. The window has opposing end edges. The button element and the window are separate components. The button element has opposing end edges that align with the end edges of the window where the button element and window meet to provide an impression that the window and the button element are integral with one another.

Aspect 25 concerns a method of manufacturing a medical device. An electronic circuit board is positioned in a back shell. The back shell has a button cavity. The electronic circuit board includes a display and a switch. The switch is aligned to face the button cavity. A button element is inserted into the button cavity. The button element has opposing end edges. The button element includes an actuator structure configured to actuate the switch. The actuator structure is aligned to face the switch of the electronic circuit board. A front shell is attached to the back shell to retain the button element in the button cavity. The front shell includes a window having opposing end edges. The window is positioned to cover at least a portion of the display in which the display is visible through the window. The end edges of the window are aligned with the end edges of the button element where the window meets the button element.

Aspect 26 concerns the method of any preceding aspect, in which the button element has a stiffening rib which extends along a front shell facing edge. The front shell facing edge is positioned to face the front shell when the front shell is attached to the back shell.

Aspect 27 concerns the method of any preceding aspect. The back shell has a button alignment structure that has a groove. The button element has an alignment rib that extends transverse to the stiffening rib. The alignment rib is slid into the groove.

Aspect 28 concerns the method of any preceding aspect, wherein the window includes a rounded edge. The button element has a body portion. The rounded edge of the window is aligned to be flush with the body portion of the button element.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a perspective view of one example of a glucose meter incorporating an integrated interface system.

FIG. 2 is a front view of the glucose meter with the window and front shell of the housing removed.

FIG. 3 is a perspective view of a button element found in the FIG. 1 integrated interface system.

FIG. 4 is an enlarged front view of the FIG. 3 button element when installed into the back shell of the housing of the FIG. 1 glucose meter.

FIG. 5 is a cross-sectional view of the button element shown in FIG. 4.

FIG. 6 is an enlarged cross-sectional view as taken along line 6-6 in FIG. 5.

FIG. 7 is a graph showing the relative movement of the points depicted in FIG. 6.

FIG. 8 is a graph showing the number of actuation cycles per force applied to the FIG. 3 button element.

FIG. 9 is a perspective view of a glucose meter incorporating another example of an integrated interface system.

FIG. 10 is a front view of the FIG. 9 glucose meter with the window and front shell of the housing removed.

FIG. 11 is a perspective view of an interior side of the button element found in the FIG. 9 window-button system.

FIG. 12 is a perspective view of an exterior side of the FIG. 11 button element.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

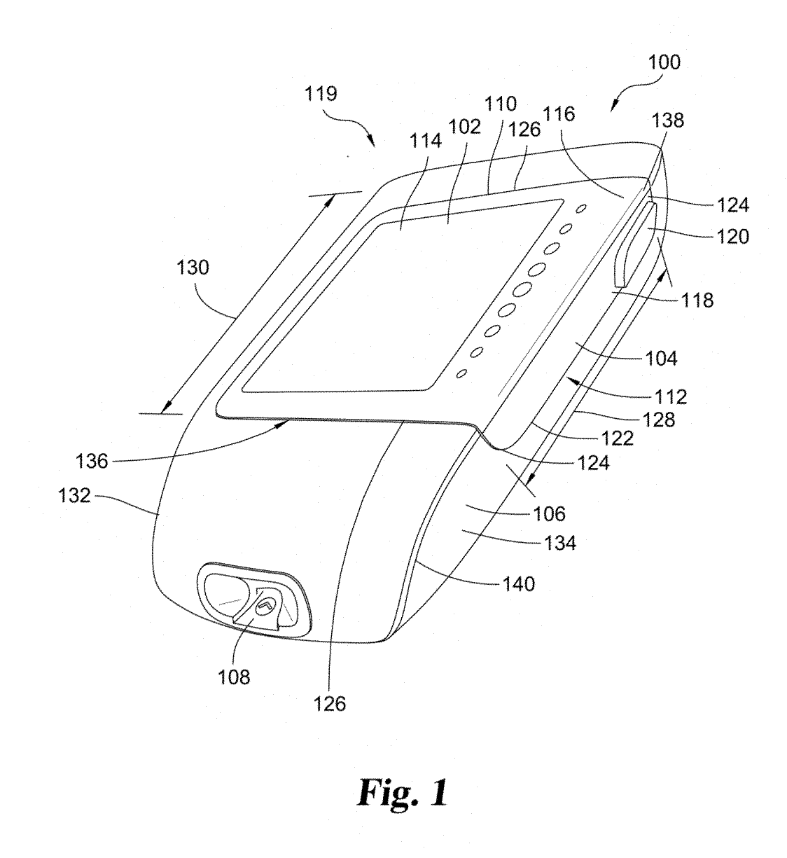

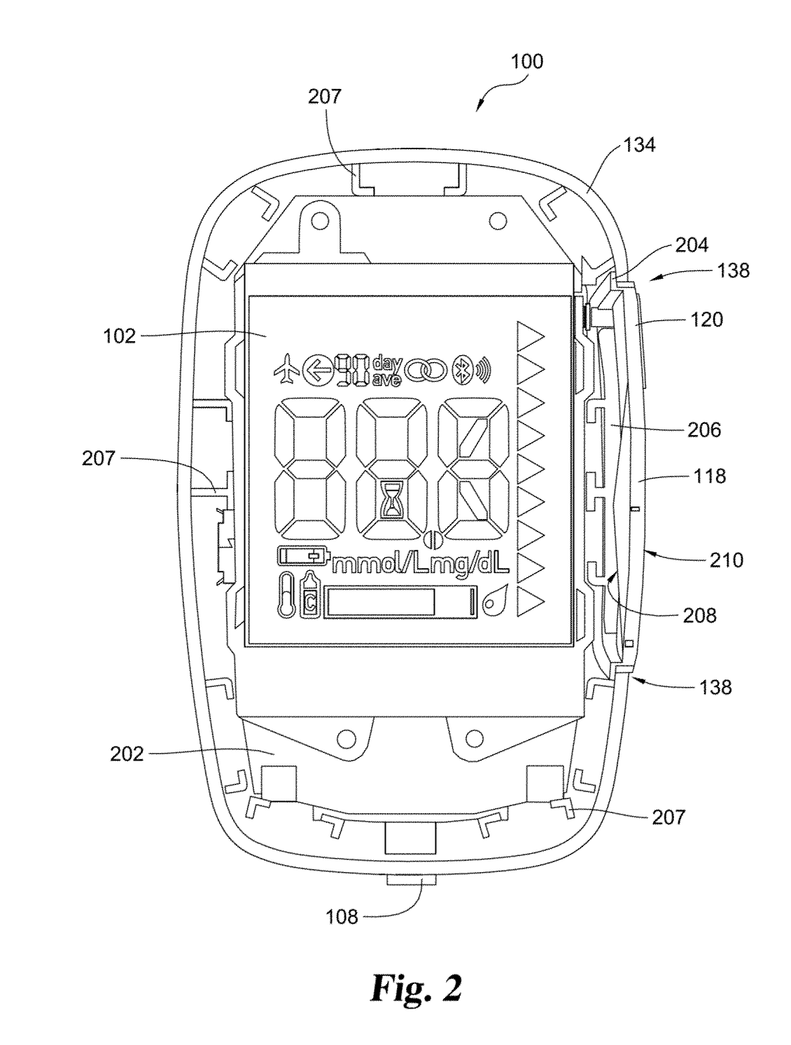

The reference numerals in the following description have been organized to aid the reader in quickly identifying the drawings where various components are first shown. In particular, the drawing in which an element first appears is typically indicated by the left-most digit(s) in the corresponding reference number. For example, an element identified by a “100” series reference is numeral will likely first appear in FIG. 1, an element identified by a “200” series reference numeral will likely first appear in FIG. 2, and so on.

FIG. 1 shows a perspective view of a medical diagnostic device in the form of a glucose meter 100 that includes a display 102 for viewing test results as well as other information and an input device 104 for interacting with the glucose meter 100. The glucose meter 100 further includes a housing 106 and a test strip connector port 108 that is configured to receive a test strip. As shown, the display 102 is positioned on a front face 110 of the housing 106, and the input device 104 is positioned on a side face 112 of the housing 106. In the illustrated example, the side face 112 is angled at an acute angle relative to the front face 110, but in other examples, the front 110 and side 112 faces can be generally oriented at a right angle. The display 102 is covered and protected by a display window or lens 114. The window 114 is transparent or semitransparent so that the user can see the display 102. In one form, the window 114 is made of a transparent plastic material. Along the side face 112 of the meter 100, the window 114 has a rounded edge or bevel 116 so as to provide an illusion of the window 114 bending from the front face 110 to the side face 112 in a waterfall type manner. The input device 104 in the illustrated example is in the form of a button element or strip 118. The button element 118 in one form is made from a flexible plastic. Together, the window 114 and the button element 118 form an integrated interface system 119 that provides an integrated appearance. To further provide the impression that the button element 118 and the window 114 are a single unit, the button element 118 is flush with the rounded edge 116 of the window 114. The button element 118 includes an actuation or button portion 120 that is configured to be pressed by the user and a body portion 122 that provides a visual appearance of the button element 118 being part of the window 114. In the illustrated example, the button portion 120 is raised relative to the body portion 122, but in other examples, the button portion 120 can be differentiated from the body portion 122 (or not) in other manners, such as by having different surface textures, colors, icons, or other properties. To further get the impression that the window 114 and the button element 118 in the system 119 are one, the button element 118 has end edges 124 that are located to align with end edges 126 at the rounded edge 116 of the window 114. As can be seen, the length 128 of the button element 118 (as measured from end edges 124) is generally the same as the length 130 (as measured from end edges 126) of the window 114. In addition, the window 114 and button element 118 are manufactured to have the same visual appearance. For instance, the window 114 and button is element 118 in one form have the same or similar color and surface texture so as to provide the impression that these components form the integrated interface system 119. The visual appearance of the components in the integrated interface system 119 can contrast in color with the other components, such as the housing 106, so as to further provide the illusion of being an integral unit. Such an integrated appearance also provides cues to those with visual impairment as to the location of the input device 104.

The housing 106 has a two shell design that includes a front or top shell 132 and a back or bottom shell 134 that are joined together in a snap fit type fashion or in other ways, such as via screws and/or adhesives. The front shell 132 defines a window cavity 136 in which the window 114 is received. In the illustrated example, the window cavity 136 includes a recessed area having a thickness that matches the thickness of the window 114 so that the window is flush with the front face 110 of the front shell 132. The window cavity 136 further includes a through opening inside the recess so as to frame the display 102. It is envisioned that the window cavity 136 can be shaped differently in other examples. For instance, the recess can be eliminated and/or the opening for framing the display 102 can be extended completely to the edge of the front shell 132 by the button element 118. In one form, the window 114 is attached to the front shell 132 before the front shell 132 is attached to the back shell 134. The window 114 can be attached to the front shell 132 in any number of ways, such as through an adhesive, fastener, and ultrasonic welding, to name just a few examples. In other examples, the window 114 can be integrally formed with the front shell 132 or attached after the front 132 and back 134 shells are joined together. The back shell 134 defines a button cavity 138 in which the button element 118 is received. As can be seen, the window cavity 136 and the button cavity 138 are aligned with one another such that the window 114 and button element 118 are likewise aligned with one another so as to give the impression of both being a single integral unit. When the glucose meter 100 is assembled, seams 140 between both the front 132 and back 134 shells as well as the window 114 and button element 118 are formed. The seams 140 are aligned so as to minimize the impression of the window 114 and button element 118 being separate.

FIG. 2 shows a front view of the glucose meter 100 with both the front shell 132 and window 114 removed. As shown, the display 102 is mounted on an electronic circuit board 202 that is operatively coupled to both the display 102 and the test strip connector 108. In one form, the display 102 is a liquid crystal display (LCD), but it should be recognized that the display 102 is can include other types of displays, such as light emitting diode (LED) and organic light emitting diode (OLED) type displays, to name just a few. The electronic circuit board 202 includes one or more processors and memory for processing the signals generated from a test strip once connected to the test strip connector 108 during testing and displaying the test results on the display 102. The electronic circuit board 202 further includes a switch or contact 204 that provides an interface between the user and the glucose meter 100. The switch 204 is positioned proximal to the button element 118 such that the button element 118 is able to actuate the switch 204 when pressed. Proximal to the button cavity 138, the back shell 134 has a button alignment structure 206 positioned to align the button element 118 within the button cavity 138 as well as the rest of the components in the glucose meter 100. The back shell 134 includes a series of board alignment members 207 that are positioned around the periphery of the electronic circuit board 202 so as to properly orient and align the circuit board 202 within the back shell 134. The alignment members 207 in turn help to locate the display 102 properly relative to the window 114. The alignment members 207 in certain forms can include snap connectors or other types of connecting structures for securing the electronic circuit board 202 to the back shell 134. As illustrated, the button element 118 has an interior side 208 and an exterior side 210. The interior side 208 is configured to engage the button alignment structure 206, and the exterior side 210 includes the button portion 120 for interfacing with the user. During assembly, once the front shell 132 is snapped into place onto the back shell 134, the button element 118 becomes trapped such that the button element 118 is unable to move out of the button cavity 138.

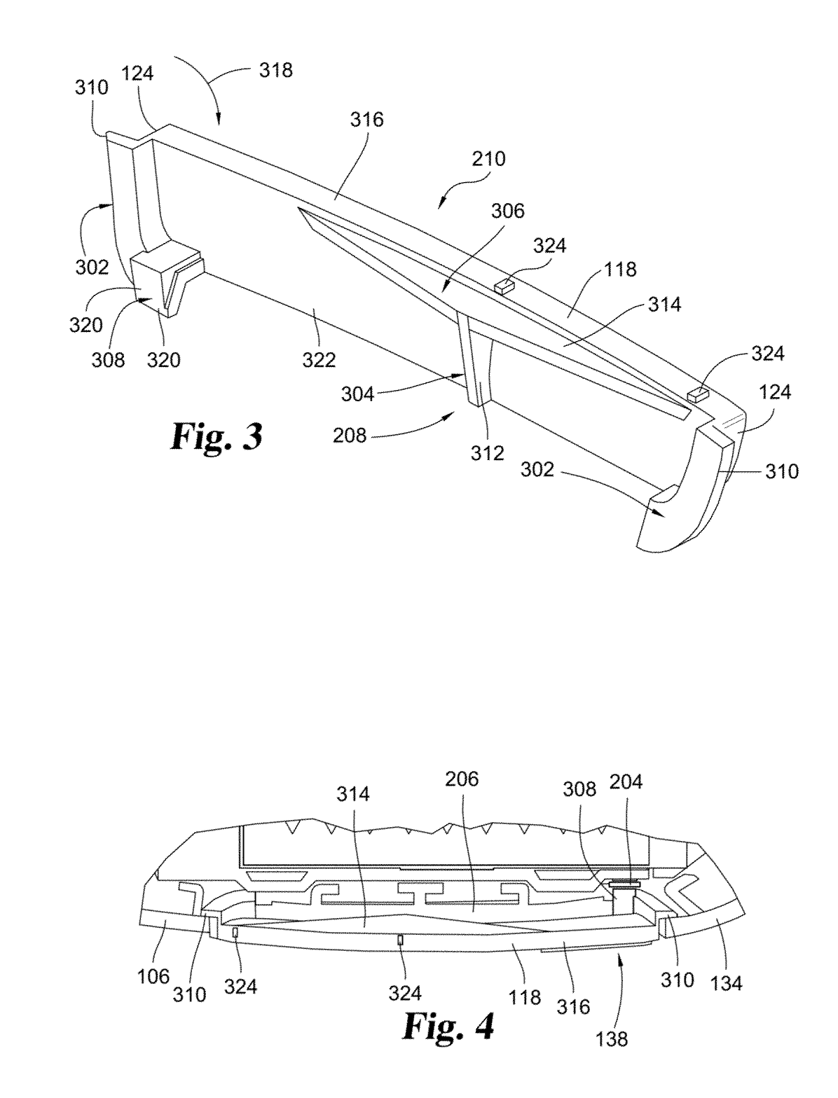

Turning to FIG. 3, a perspective view of the interior side 208 of the button element 118 is shown. In one form, the button element 118 is made of plastic and manufactured using an injection molding process, but it should be recognized that the button element 118 can be made from different materials and/or in other manners. As depicted, the button element 118 on the interior side 208 includes a retention structure 302, an alignment structure 304, a stiffening structure 306, and an actuator structure 308. The retention structure 302 is configured to retain the button element 118 within the button cavity 138 of the back shell 134. In the illustrated example, the retention structure 302 is in the form of one or more retention flanges 310 that extend from the end edges 124 of the button element 118. The exterior side 210 of the button element 118 extends from the retention flanges 310 so as to be generally flush with the rounded edge 116 of the window 114 along with the surface of the housing 106.

The alignment structure 304 is designed to align the button element 118 with the rest of the components of the glucose meter 100. The alignment structure 304 in the depicted example includes an alignment rib 312 that extends generally transverse to the length of the button element 118 near the midway point between the end edges 124. The alignment rib 312 is generally tapered so as to facilitate the button element 118 being dropped into the button cavity 138 in the back shell 134 during assembly. The alignment rib 312 is designed as a hard point to engage the button alignment structure 206 of the back shell 134 so as to ensure the button element 118 is properly positioned within the button cavity 138.

The stiffening structure 306 is configured to facilitate a biased flexing and twisting action of the button element 118 when actuated. In FIG. 3, the stiffening structure 306 is in the form of a stiffening rib 314 that extends longitudinally along the length of the button element 118. The stiffening rib 314 is offset from the central longitudinal axis of the button element 118 such that the stiffening rib 314 generally extends along a front shell facing edge 316 of the button element 118. In the illustrated example, both ends of the stiffening rib 314 are tapered so as to create a generally triangular shape. The alignment rib 312 and the stiffening rib 314 intersect to generally form a T-shaped structure. With this construction of the stiffening rib 314, the button element 118 is configured to naturally twist about its longitudinal axis, as is indicated by arrow 318 in FIG. 3, when the button portion 120 is pressed. Once the pressure against the button portion 120 is removed, the button element 118 twists back to its original shape.

With continued reference to FIG. 3, the actuator structure 308 is positioned to contact and actuate the switch 204 when the button element 118 is twisted as the button portion 120 is pressed. In the illustrated example, the actuator structure 308 is in the form of an actuator foot 320. The actuator foot 320 extends from the interior side 208 of the retention flanges 310 that is located at the same end of the button element 118 as the button portion 120. Moreover, the actuator foot 320 is positioned proximal to a back shell facing edge 322 of the button element 118, which is located opposite the front shell facing edge 316. Along the front shell facing edge 316, the button element 118 includes one or more spacer tabs 324 that are configured to space the button element 118 from the front shell 132. As can be seen, the spacer tabs 324 are positioned along the end of the button element 118 that is away from the actuator structure 308. The spacer tabs 324 help to maintain a small gap in the seam 140 that allows the button element 118 to twist freely without binding with the front shell 132.

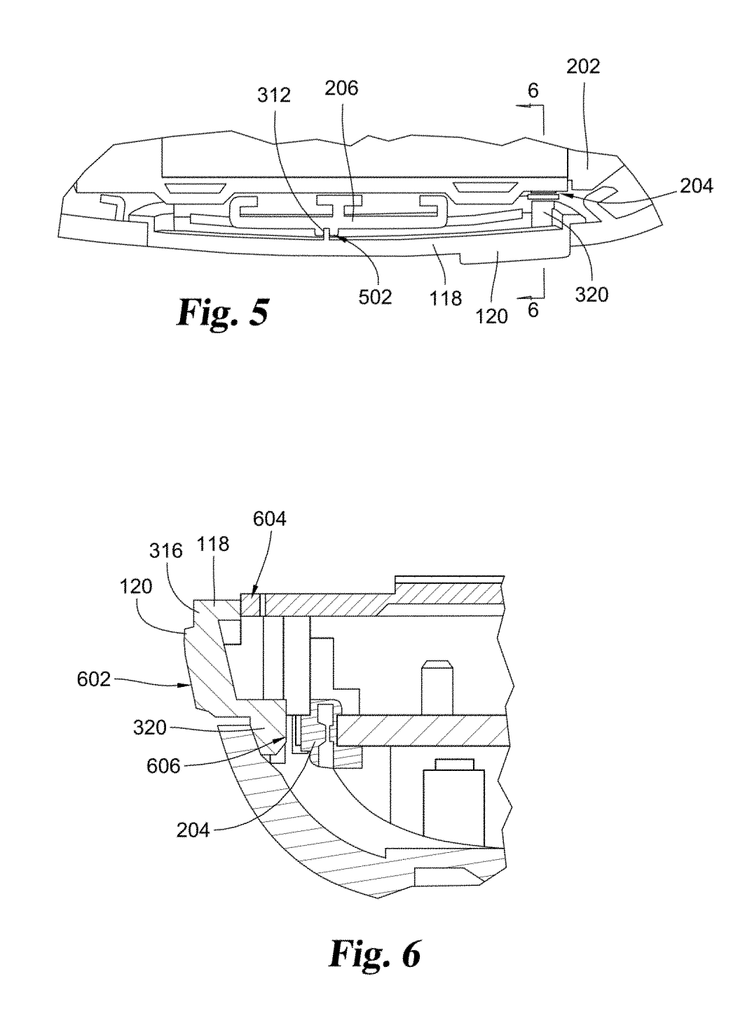

FIG. 4 shows an enlarged front view of the button element 118 when installed in the button cavity 138 of the back shell 134. As can be seen, the retention flanges 310 engage the walls of the back shell 134 of the housing 106. In one form, the stiffening rib 314 can act as a stop to prevent over insertion of the button element 118. Turning to FIG. 5, the alignment rib 312 of the button element 118 is received in an alignment groove 502 defined in the button alignment structure 206. Again, the alignment rib 312 ensures the button element 118 is properly positioned such that the actuator foot 320 is positioned properly to actuate the switch 204 on the circuit board 202. Rather than bending at a single, concentrated location, which can lead to premature failure, the button element 118 is designed to flex and twist along most of its length so as to disperse the stress of the load. This twisting action can extend the operational life of the button element 118. Specifically, when the user of the glucose meter 100 presses the button portion 120, the button element 118 twists such that the actuator foot 320 pushes against the switch 204 so as to create a signal. The resulting signal is used as input for the electronic circuit board 202. For example, pressing the button portion 120 can toggle between various readouts, menus, and screens shown on the display 102 of the glucose meter 100.

FIG. 6 shows a cross-sectional view of the switch 204 and the button element 118 as taken along line 6-6 in FIG. 5. FIG. 6 shows various points of movement on the button element 118 as the button portion 120 is pressed. Reference point “A” 602 is located generally on the button portion 120. Reference point “B” 604 is generally located at the stiffening rib 314 which is proximal to the front shell facing edge 316 of the button element 118, and reference point “C” 606 is positioned at the surface of the actuator foot 320 where contact is made with the switch 204. As alluded to before, the stiffness of the stiffening rib 314 is adjusted or selected to obtain simultaneous movement of reference points A 602, B 604, and C 606 during actuation of the button element 118. FIG. 7 includes a movement analysis graph 700 showing the relative movement of reference points A 602, B 604, and C 606 during such actuation. As can be seen in the graph 700, the movement of these points is generally simultaneous.

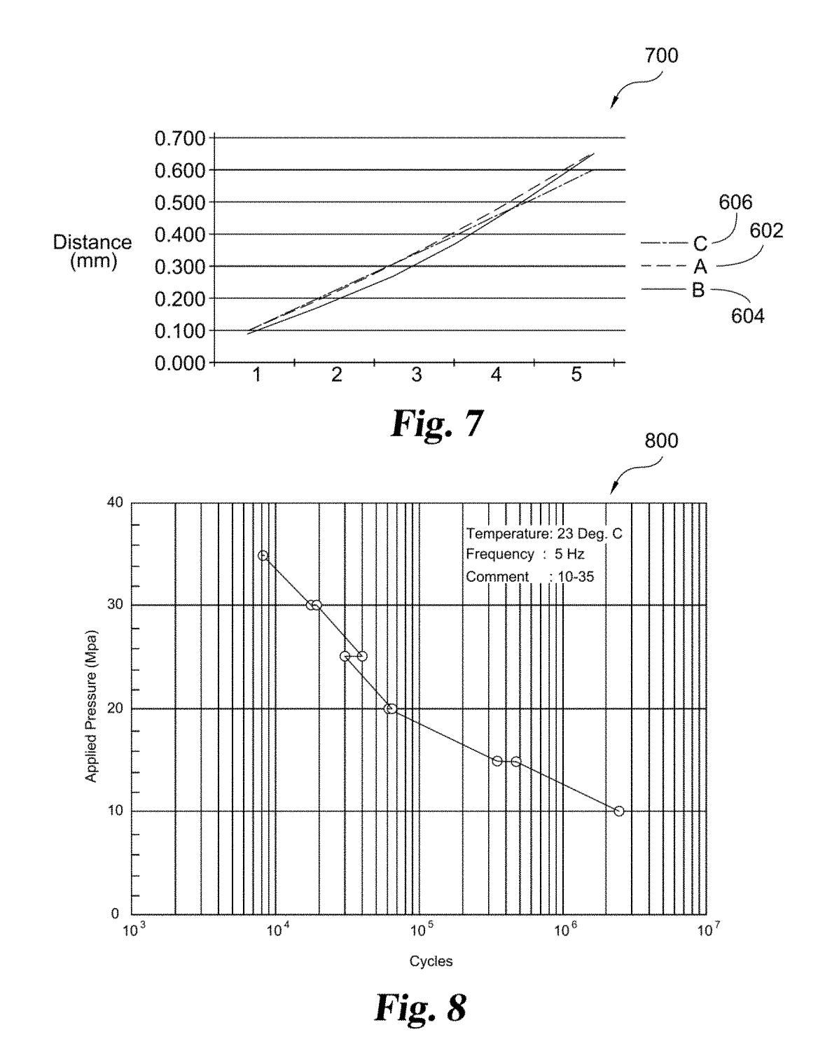

This construction of the button element 118 provides a robust input device that can be used over multiple cycles (i.e., pressed and released). FIG. 8 shows a simulated fatigue curve graph 800 for the button element 118 based on the pressure applied against the button portion 120. As shown, based on the fatigue curve at 11.3 megapascals (Mpa) of applied pressure to the button portion 120, the button element 118 can survive 1×10⁶ cycles which enhances the operational life for the input device 104 of the glucose meter 100. As should be recognized, the integrated interface system 119 provides a robust input device that is easy to assemble while also making the glucose meter 100 aesthetically interesting. The construction of the glucose meter 100 is also simplified because the need for a biasing spring is eliminated.

A method of manufacturing the glucose meter 100 will now be described with reference to FIGS. 1-6. Looking at FIG. 2, the electronic circuit board 202 is positioned in the back shell 134. As noted before, the board alignment members 207 orient the circuit board 202 within the back shell 134. As can be seen, the switch 204 on the circuit board 202 is positioned to face the button cavity 138. Before, during, or after the electronic circuit board 202 is placed in the back shell 134, the button element 118 is inserted in the button cavity 138 such that the flanges 310 engage the wall of the back shell 134 surrounding the button cavity 138, as is depicted in FIG. 4. During insertion of the button element 118, the alignment rib 312 slides within the alignment groove 502 (FIG. 5) of the button alignment structure 206, and the retention flanges 310 engage the walls of the back shell 134 to hold the button element 118 in place, thereby countering the force from the alignment rib 312 that is pushing the button element 118 out of the button cavity 138. This arrangement helps to ensure the actuator foot 320 properly faces the switch 204 of the electronic circuit board 202. By extending along the front facing edge 316 and extending transverse to the alignment rib 312, the stiffening rib 314 in FIG. 4 prevents the button element 118 from being inserted upside down. Consequently, the front shell facing edge 316 of the button element 118 is ready to be positioned to face the window 114 in the assembled glucose meter 100. Once the button element 118 is properly positioned within the button cavity 138, the front shell 132 is attached to the back shell 134 such that the button element 118 is enclosed and retained within the button cavity 138. During the attachment of the front shell 132 to the back shell 134, the window 114 is positioned over the display 102 so that all or most of the display 102 is visible through the window 114. The front shell 132 is positioned such that the end edges 126 of the window 114 are aligned with the end edges 124 of the button element 118 so as to provide the visually integral appearance between the window 114 and the button element 118. The front shell 132 and the back shell 134 can be secured together in any number of manners. For example, the front shell 132 and the back shell 134 can be secured together through snap fit connections, fasteners, adhesives, ultrasonic welding, and interference fitting, to name just a few examples.

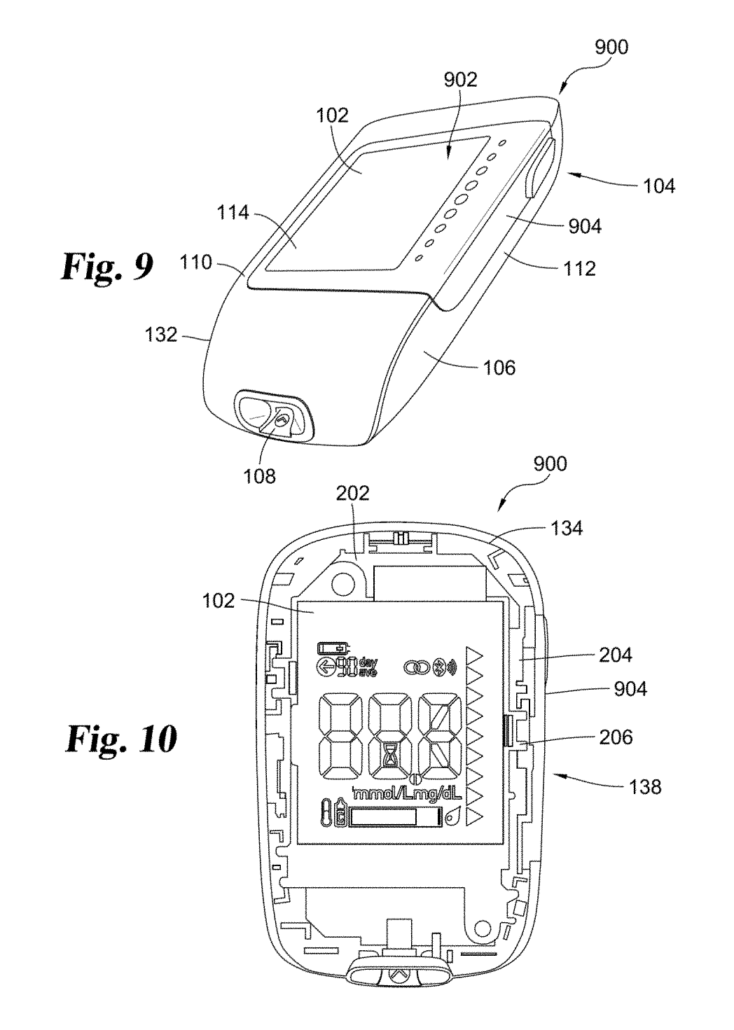

FIGS. 9 and 10 illustrate a medical diagnostic device in the form of a glucose meter 900 that includes another example of an integrated interface system 902. Like the previous example, the integrated interface system 902 is configured to give the visual impression that the display 102 and the input device 104 form an integrated unit that provides an overall waterfall style design in which the window 114 appears to bend from the front face 110 to the side face 112 of the glucose meter 900. The glucose meter 900 in FIGS. 9 and 10 shares most of the components in common with the glucose meter 100 described with reference to FIGS. 1-8. For example, the glucose meter 900 includes the display 102, housing 106, test strip connector 108, window 114, housing shells 132, 134, circuit board 202, and switch 204 in common with the one described before. For the sake of clarity as well as brevity, these common components will not be described in detail again, but reference is made to the previous discussion of these common components. Likewise, the glucose meter 900 in FIG. 9 can be manufactured using the same or similar methods of those described above with respect to the glucose meter 100 of FIG. 1. In the illustrated example, the integrated interface system 902 includes a button element 904 that is configured somewhat differently than the button element 118 described previously with respect to FIGS. 1-8. Like the previous example, the button element 904 is configured to be received in the button cavity 138 in the back shell 134.

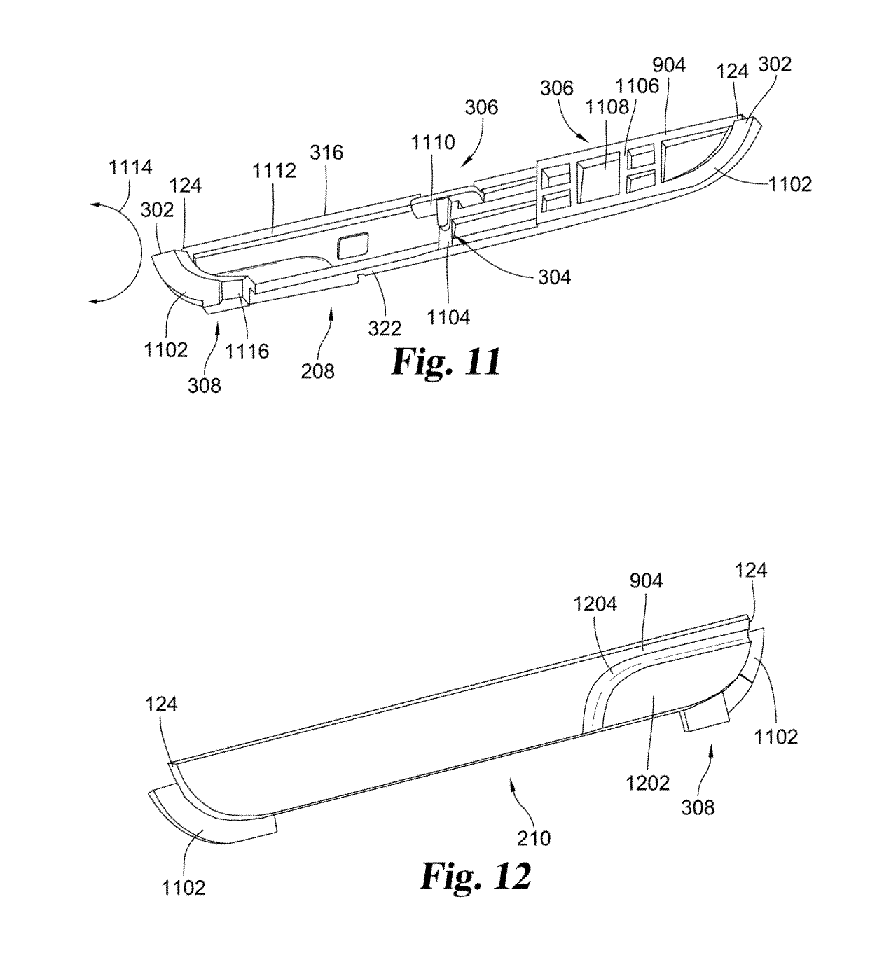

The button element 904 is illustrated in further detail in FIGS. 11 and 12. FIG. 11 shows a perspective view of the button element 904 from the interior side 208, and FIG. 12 shows the button element 904 from the exterior side 210. Generally similar to the previous example, the button element 904 in FIG. 11 includes retention 302, alignment 304, stiffening 306, and actuator 308 structures. The retention structure 302 in the illustrated example includes retention flanges 1102 positioned at the opposite end edges 124 of the button element 904. The retention flanges 1102 are configured to engage the interior side of the back shell 134 surrounding the button cavity 138 so as to retain the button element 904 in the housing 106. The alignment structure 304 in the illustrated example includes an alignment rib 1104. The alignment rib 1104 has a generally rounded end and tapered sidewalls so as to facilitate insertion into the button alignment structure 206 in the back shell 134 (FIG. 10). This alignment rib 1104 is configured to readily align the button element 904 when dropped into the button cavity 138 of the housing 106 during assembly.

The stiffening structure 306 in the FIG. 11 button element 904 includes several structures for facilitating the twisting action of the button element 904 when pressed or otherwise actuated. The stiffening structure 306 is oriented asymmetrically along the button element 904 so as to promote the twisting action of the button element 904 when actuated. At the end opposite the actuator structure 308, the stiffening structure 306 includes a lattice of ribs 1106 that define rib cavities 1108 so as to reduce the overall weight of the button element 904. The lattice of ribs 1106 stiffen the button element 904 at the end that is opposite the actuator structure 308. The stiffening structure 306 in FIG. 11 further includes a first stiffening rib 1110 that is oriented to form a T-shaped relationship with respect to the alignment rib 1104. The first stiffening rib 1110 is positioned proximal to the front shell facing edge 316 such that the first stiffening rib 1110 is offset from the longitudinal axis of the button element 904. A second stiffening rib 1112 extends along the front shell facing edge 316 towards the actuator structure 308. These various stiffening structures 306 promote a twisting action of the button element 904 when pressed and released, as is indicated by double arrow 1114 in FIG. 11.

In the illustrated example, the actuator structure 308 includes an actuator foot 1116 extending from one of the retention flanges 1102. The actuator foot 1116 extends from the retention flanges 1102 and is positioned so as to contact the switch 204 when the button element 904 is pressed. Turning to FIG. 12, the actuator foot 1116 is located opposite (i.e., on the interior side 208) from an actuation or button portion 1202. In one form, the button portion 1202 is raised from the rest of the button element 904 on the exterior side 210. In order to provide a further visual indicator, the periphery of the button portion 1202 is surrounded by a groove 1204. To actuate the switch 204, the user presses the button portion 1202 with their finger or thumb, for example. The resulting pressure causes the button element 904 to twist (arrow 1114), and the twisting action in turn causes the actuator foot 1116 to press against and close the switch 204. Again, closing or otherwise activating the switch 204 can be used to cause a number of different actions within the glucose meter 900. For example, pressing the button portion 1202 can cause the glucose meter 900 to toggle through various screens on the display 102 and/or change numerous operational modes. It can also be used to silence any types of alerts or alarms, to name just a few use examples. The illustrated integrated interface system 902 in FIG. 9 provides an aesthetically pleasing as well as functionally robust system for interfacing with the glucose meter 900. Moreover, this design helps to simplify manufacturing which makes it viable for high-volume consumer use.

Though the integrated interface systems were described above with respect to glucose is meters, it should be recognized that the systems can be in incorporated into other types of diagnostic or medical devices. In the illustrated examples, the integrated interface systems were designed for right-handed use by having the button elements located on the right-hand side of the glucose meters, but in other variations, the integrated interface systems can be adapted for left-handed use by positioning the button elements on the left side of the glucose meters or other medical devices (or even ambidextrous use). The housings, display windows, and button elements are normally made of plastic, but in other examples, these components can be made from other types of materials and in different combinations of materials. The window and button elements are described above as having the same or similar color and/or other visual appearance at least where they meet. It should be recognized that the window and button elements can have the same visual appearance throughout their entirety or the similarity can be just limited to where they meet. For example, in one variation, the shade of the window gradually changes towards the button element such that the shade of both components where the window and button element meets is the same, but at locations farther away from the button element, the window has a different shade than the button element. In the examples described above, the rounded edge 116 of the window is generally flush with the button element where both components meet (i.e., at the seam). However, in other examples, these components might not be entirely flush where they meet. For instance, a protruding button portion may extend all the way towards the seam such that this portion of the button element would not be flush with the rounded edge, but most of the button element would be flush. As can be seen in the drawings, the window and button element have similar rounded edges that further aid in the visual impression that both components are integral with one another or the same. It should be appreciated that other visual indicators can further facilitate this visual impression that both components are integral. The window can be secured to the front shell of the housing in any number of manners, such as through adhesives, ultrasonic welding, etc., and the shells of the housing can be secured in any number of manners, such as through snap fit connections, adhesives, ultrasonic welding, fasteners, or combinations thereof. In the illustrated examples, the stiffening rib is positioned along the front shell facing edge of the button on and the actuator foot is positioned along the back shell facing edge so as to promote the flexing or twisting action. In other examples, the stiffening rib can be positioned along the back shell facing edge and the actuator foot can be positioned along the front shell facing edge such that the button element twists or flexes an opposite fashion.

Though directional terms, such as front, back, interior, exterior, etc., were used to describe the design above, it should be appreciated that the invention should not be limited to specific directional orientations. While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A medical device, comprising:
a housing having a front face and a side face, the housing defining a window cavity on the front face and a button cavity in the side face;
a display disposed in the housing, the display being aligned in the housing for viewing through the window cavity;
a window covering the display in the window cavity, the display being at least partially visible through the window, the window extending along the front face to the button cavity in the side face, the window having opposing end edges; and
a button element received in the button cavity, the button element and the window being separate components, the button element having opposing end edges that align with the end edges of the window where the button element and the window meet to provide an impression that the window and the button element are integral with one another.

2. The device of claim 1, wherein:
the button element has an exterior surface; and
the window has a rounded edge that at least a portion of which is flush with the exterior surface of the button element where the window and button element meet to provide the impression that the window bends from the front face to the side face.

3. The device of claim 2, wherein the button element has a button portion that protrudes from the exterior surface of the button element.

4. The device of claim 1, wherein the window and the button element have similar visual appearances.

5. The device of claim 4, wherein the window and the button element at least where the window and the button element meet have the same color.

6. The device of claim 1, wherein the button element includes a stiffening structure asymmetrically positioned along a longitudinal axis of the button element to promote a twisting motion of the button element when pressed.

7. The device of claim 6, wherein the stiffening structure includes a stiffening rib positioned proximal to a side of the button element facing the window.

8. The device of claim 7, wherein the button element includes an alignment rib extending transverse to the stiffening rib to form a T-shape.

9. The device of claim 6, wherein the stiffening structure includes a lattice of ribs defining rib cavities.

10. The device of claim 1, wherein:
the housing includes a button alignment structure defining an alignment groove; and
the alignment structure of the button element includes an alignment rib received in the alignment groove.

11. The device of claim 1, wherein:
the housing includes
a front shell defining the window cavity, and
a back shell defining the button cavity; and
the front shell and the back shell are coupled together to trap the button element in the button cavity.

12. The device of claim 11, wherein the button element includes one or more retention flanges engaging the button shell around the button cavity.

13. The device of claim 12, further comprising:
a switch; and
wherein the button element includes an actuator foot extending from one of the retention flanges to actuate the switch.

14. The device of claim 13, wherein:
the button element has an interior side that faces an interior of the housing and an exterior side that is opposite to the interior side;
the actuator foot extends from the interior side of the button element;
the button element has a button portion protruding on the exterior side; and
the button portion is aligned to be opposite the actuator foot.

15. The device of claim 11, wherein a seam between the front shell is aligned with a seam between the window and the button element.

16. The device of claim 11, wherein the button element has one or more spacer tabs extending into a seam between the window and the button element.

17. The device of claim 1, wherein the housing includes a test strip connector port.

18. A medical device, comprising:
a housing having a side face that defines a button cavity; and
a button element retained in the button cavity, the button element having a stiffening rib located at an offset position along the length of the button element to induce a twisting motion in the button when pressed.

19. The device of claim 18, wherein the button element includes an alignment rib extending transverse to the stiffening rib to form a T-shape.

20. The device of claim 19, wherein:
the housing includes a button alignment structure defining an alignment groove; and
the alignment rib is received in the alignment groove.

21. The device of claim 18, wherein the button element includes a lattice of ribs defining rib cavities.

22. The device of claim 18, wherein:
the button element includes a first edge and a second edge located opposite to the first edge;
the button element has an actuator foot positioned along the first edge at one end of the button element, the actuator foot extending from an interior surface of the button element;
the button element has a button portion protruding from an exterior surface at a position aligned with the actuator foot; and
the stiffening rib extends along the second edge of the button element.

23. The device of claim 18, wherein the button element includes one or more retention flanges engaging the housing around the button cavity.

24. The device of claim 18, further comprising:
wherein the housing has a front face with a window;
wherein the window extends along the front face to the button cavity in the side face, the window having opposing end edges; and
wherein the button element and the window are separate components, the button element having opposing end edges that align with the end edges of the window where the button element and window meet to provide an impression that the window and the button element are integral with one another.

25. A method of manufacturing a medical device, comprising:
positioning an electronic circuit board in a back shell, wherein the back shell has a button cavity, wherein the electronic circuit board includes a display and a switch, wherein said positioning includes aligning the switch to face the button cavity;
inserting a button element into the button cavity, wherein the button element has opposing end edges, wherein the button element includes an actuator structure configured to actuate the switch, wherein said inserting includes aligning the actuator structure to face the switch of the electronic circuit board; and
attaching a front shell to the back shell to retain the button element in the button cavity, wherein the front shell includes a window having opposing end edges;
wherein said attaching includes
positioning the window to cover at least a portion of the display in which the display is visible through the window, and
aligning the end edges of the window with the end edges of the button element where the window meets the button element.

26. The method of claim 25, further comprising:
providing the button element with a stiffening rib extending along a front shell facing edge; and
wherein said inserting the button element includes positioning the front shell facing edge to face the front shell when the front shell is attached to the back shell.

27. The method of claim 26, further comprising:
providing the back shell with a button alignment structure that has a groove;
providing the button element with an alignment rib that extends transverse to the stiffening rib; and
wherein said inserting the button element includes sliding the alignment rib into the groove.

28. The method of claim 25, wherein:
the window includes a rounded edge;
the button element has a body portion; and
said attaching includes aligning the rounded edge of the window to be flush with the body portion of the button element.
